(19) United States
(12) Patent Application Publication
(10) Pub. No.: US 2023/0067224 A1
(43) Pub. Date: Mar. 2, 2023

(54) ELECTROSURGICAL GENERATOR HAVING AN INVERTER WITH IMPROVED DYNAMIC RANGE

(71) Applicant: OLYMPUS WINTER & IBE GMBH, Hamburg (DE)

(72) Inventors: Jelle DIJKSTRA, Berlin (DE); Thomas FÄHSING, Berlin (DE); Daniel RAMIN, Nuthetal (DE)

(73) Assignee: OLYMPUS WINTER & IBE GMBH, Hamburg (DE)

(21) Appl. No.: 17/892,611

(22) Filed: Aug. 22, 2022

Related U.S. Application Data
(60) Provisional application No. 63/237,395, filed on Aug. 26, 2021.

(51) Int. Cl. H02M 7/5387; H02M 7/483; A61B 18/12

(52) U.S. Cl. CPC H02M 7/53871; H02M 7/483; A61B 18/1206; A61B 2018/00767

(57) ABSTRACT

An electrosurgical generator for an electrosurgical instrument includes DC voltage supply and high-voltage inverter that generates high-frequency AC voltage having variable voltage and frequency. Inverter is multilevel inverter controlled by reference signal and having at least two groups of series-connected inverter cells, wherein each group is supplied with different DC voltage and wherein the voltages output by the two groups are summed to be output at output. Group supplied with higher voltage enables fast and large voltage changes with its inverter cells, while inverter cells of other group supplied with lower voltage allow fine setting with high change speed. Dynamic range is improved both from temporal viewpoint and in terms of increased voltage span. The number of HVC cells to be switched may furthermore be varied by way of modulator, wherein further number of LVC cells are switched in an opposing manner for compensation purposes. Switching losses may be reduced.

The invention relates to an electrosurgical generator that is designed to output a high-frequency AC voltage to an electrosurgical instrument. It comprises a DC voltage supply and a high-voltage inverter that is fed from the DC voltage supply and generates a high-frequency AC voltage that is applied at an output for the connection of the electrosurgical instrument.

In electrosurgery, high-frequency AC current is used in particular to cut or slice through tissue and to excise body tissue within the meaning of a thermal resection (what is known as an electrical scalpel). The operating principle is based on heating the tissue to be cut. One advantage of this is that, at the same time as the cutting, it is also possible to stem bleeding by closing the affected vessels (coagulation). Considerable powers are required for this purpose, specifically at frequencies of 200 kHz or higher up to 4000 kHz, typically around 400 kHz. Body tissue behaves like an ohmic resistance at these frequencies. However, the specific resistance is strongly dependent on the type of tissue, meaning that the specific resistances of muscles, fat or bones differ greatly from one another, specifically by up to a factor of 1000. This means that, during operation, the load impedance of the electrical scalpel may change quickly and greatly depending on the tissue to be cut, as far as a virtual short circuit. This places special and unique requirements on the electrosurgical generator and its high-voltage supply. There is in particular a need for fast voltage control, suitable for high voltages in the range of a few kilovolts, and high frequencies in a wide range from typically between 200 kHz and up to 4 MHz.

Depending on the tissue and the resulting impedance, the currents vary between a few milliamperes and several amperes, specifically in a highly dynamic manner within a very short time. The waveform of the AC voltage that is output may be continuously sinusoidal or may be modulated with a crest factor of up to 10 at modulation frequencies of up to around 20 kHz.

Conventional electrosurgical generators are often structured in accordance with the principle of a single-ended high-voltage converter. They have an inverter for supplying power to the electrosurgical instrument, to which rectified current is supplied from the grid with a differing voltage. For this purpose, it is necessary for the DC supply to be able to be adjusted with regard to the DC voltage that is delivered. The inverter is typically designed as a freely oscillating single-ended generator having an LC resonant circuit (by way of example: EP 2514380 B1). This type of structure is proven in practice but also exhibits disadvantages. First of all, efficiency is low due to high losses. In addition, large reactive currents occur in the resonant circuit, thereby necessitating larger components and additionally worsening efficiency at low power. Furthermore, the output frequency is load-dependent, as is the crest factor, this being inappropriate for highly modulated modes. The regulation of the output voltage is comparatively slow, meaning that matching to changed load impedances is only poor.

In order to better meet these unique requirements, the applicant has developed a previously unpublished novel concept for electrosurgical generators using a multilevel inverter. This allows improved controlled generation and outputting of the AC voltage for the electrosurgical instrument. A complex DC supply with a changing voltage is no longer necessary here, but rather the value (and frequency and waveform) of the output AC voltage is controlled directly via the multilevel inverter. However, the dynamic range is limited, specifically in particular at a lower output AC voltage amplitude. The achievable modulation index decreases as the output voltage decreases. This limits not only the ability to output modulated signal forms (what are known as modes), but potentially worsens the waveform of the generated AC voltage as well.

It would be possible to counter this by making the value of the DC voltage supply of the inverter changeable. However, this thus results in the loss of the advantage of a supply with a constant voltage and the accompanying simplification of the DC voltage supply.

The invention is based on the object of providing an electrosurgical generator that provides a higher dynamic range without having to rely on a DC voltage supply with a changeable voltage value.

The solution according to the invention lies in the features of the independent claim. Advantageous developments are the subject of the dependent claims.

In the case of an electrosurgical generator that is designed to output a high-frequency AC voltage to an electrosurgical instrument, comprising a DC voltage supply and a high-voltage inverter that is fed from the DC voltage supply and generates a high-frequency AC voltage having a variable voltage and frequency that is applied to an output for the connection of the electrosurgical instrument, provision is made, according to the invention, that the inverter is designed as a multilevel inverter controlled by a reference signal for the voltage to be output and having at least two groups of series-connected inverter cells, wherein each group is supplied with a different DC voltage and wherein the voltages output by the groups are summed to be output at the output.

The invention is essentially based on the concept of using inverter cells having a different DC voltage supply. This offers the advantage of being able to set a significantly higher number of voltage levels using the same number of inverter cells (than is able to be set using the same number of inverter cells supplied with an identical DC voltage), such that the same number of inverter cells is used to achieve a better curve form of the output voltage, and this is also additionally linked to the advantage of the better dynamic range.

By virtue of the division into these at least two groups, the invention combines two advantages that appear to be contradictory, specifically firstly of enabling fine setting of even small voltages or voltage differences at a high speed by switching the inverter cells of the group supplied with a lower voltage (LVC), and secondly of enabling fast and large voltage jumps by switching the inverter cells of the group supplied with a higher voltage (HVC). The invention thus creates a two-channel structure, so to speak, having a heavy-duty channel, in which the inverter cells of the group supplied with a higher voltage (HVC) are arranged, and having a dynamic channel in which the inverter cells of the group supplied with a lower voltage (LVC) are arranged. By virtue of clocking the inverter cells of the group supplied with a lower voltage (LVC) frequently and in a finer manner with respect to the voltage levels, it is possible to achieve a finer resolution even at low voltages, whereas, by virtue of switching the inverter cells of the group supplied with a higher voltage (HVC), it is possible to quickly set even large voltages and/or large voltage jumps. This thus achieves an improvement in the dynamic range not only in relation to a temporal change, but also in relation to the voltage span and the voltage value. The arrangement according to the invention is thus also suitable for generating modulated voltage signals, as are important in particular for what are known as modes in electrosurgical generators.

The inverter cells are preferably divided into at least two groups, of which a first group (LVC) are supplied with a lower DC voltage than a different, second group (HVC). In this sense, the first group comprises low-voltage inverter cells and the second group comprises higher-voltage inverter cells. This enables fast setting of even large voltages, by appropriately actuating the higher-voltage inverter cells (HVC). The low-voltage inverter cells (LVC) by contrast offer the advantage that, due to their lower supply voltage, the switching losses of the low-voltage inverter cells (LVC) are disproportionately lower, and these thus tolerate frequent switching considerably better than the higher-voltage inverter cells (HVC), and lower switching losses also occur in this case. If for example the low-voltage inverter cells (LVC) are designed for a voltage of just one quarter of the higher-voltage inverter cells (for example for 12 V for LVC instead of 48 V for HVC), then the switching losses of the low-voltage inverter cells (LVC) contribute only one sixteenth of the switching losses of the higher-voltage inverter cells (HVC). The low-voltage inverter cells (LVC) are thus particularly suitable not only for finely setting the voltage to be output, but rather this may also take place extremely quickly. It will be understood that provision may also be made for other groups whose inverter cells are preferably supplied with other DC voltages that are also different.

The groups preferably comprise a first group whose inverter cells are supplied with a lower DC voltage (LVC) than the inverter cells (HVC) of a different, second group.

An explanation is given below of a few terms that are used:

In the field of electrosurgical generators, “high-frequency” frequencies are typically understood to be in the range from 200 kHz to 4000 kHz. Optionally, in advantageous embodiments, the ultrasonic range may also be covered. The ultrasonic range is understood to mean a frequency range between 20 kHz and 200 kHz.

“High-voltage” is typically understood to mean voltages up to 10 kV, preferably up to 5000 V.

The power provided by electrosurgical generators is typically in the range between 1 and 500 watts, wherein the load impedance may vary greatly, and output voltage and power output may accordingly likewise change greatly and quickly.

A “different” DC voltage is understood to mean a DC voltage having a different voltage absolute value; a simple reversal of polarity is not a different DC voltage in this sense.

The terms activate/switch on or deactivate/switch off are used here for the inverter cells as a synonym for increasing the voltage output by inverter cells by one level or reducing it by one level.

Expediently, the inverter cells have a bipolar configuration and are designed to output at least three different output voltage levels, specifically positive, negative and zero. It is thereby possible to use simple means to achieve an increase in the number of voltage levels and furthermore also symmetrical, bipolar generation of the AC voltage. It is also possible, by “reversing the polarity” of the inverter cells, that is to say by changing the output voltage over from positive to negative and vice versa, in the case of the output voltage of the respective inverter cells, to change the voltage by two levels in one go, when it switches from positive to negative (or vice versa).

Provision is furthermore advantageously made that there is a fixed ratio between the absolute value of the voltage that is generated by the individual higher-voltage inverter cells (HVC) and the absolute value of the voltage that is generated by the individual lower-voltage inverter cells (LVC). This may expediently be achieved by designing the DC voltage supply of the two groups of inverter cells to be linked. It is particularly advantageous here for the voltage supply for the lower-voltage inverter cells (LVC) to be generated from the DC voltage source for the higher-voltage inverter cells (HVC) by way of a ratiometric voltage converter. On the one hand, this thus requires only one actual DC voltage source. On the other hand, this thus means that the DC voltage for the higher-voltage inverter cells (HVC) is in a fixed ratio with the DC voltage, thus the voltage output for the low-voltage inverter cells (LVC). It is particularly preferable here for the ratio between the DC voltage sources for the inverter cells of the two groups to be an integer voltage multiple. A plurality, specifically a number corresponding to the voltage multiple, of series-connected lower-voltage inverter cells (LVC) thus yield exactly the voltage level of one higher-voltage inverter cell (HVC). This makes it possible, in the event of voltage changes, to switch, instead of multiple lower-voltage inverter cells (LVC), one higher-voltage inverter cell (HVC) (possibly connected to further lower-voltage inverter cells—LVC), and thus also to bring about larger voltage jumps with a lower number of switching procedures. It has been shown to be expedient for the integer voltage multiple to be at least four.

In order to drive the inverter cells of both groups, provision is expediently made for a modulator that is designed to reduce switching frequencies of the high-voltage inverter cells (HVC) by selectively replacing actuation of the high-voltage inverter cells (HVC) with actuation of a plurality of the low-voltage inverter cells (LVC). The invention makes use of the finding here that switching of the higher-voltage inverter cells (HVC) is a considerably greater load for these cells (in comparison with the load on the low-voltage inverter cells —LVC), and in this case leads to significantly greater switching losses than in the case of switching lower-voltage inverter cells (LVC). Surprisingly, this is also the case when the same voltage (for example 48 V), instead of being brought about by switching a higher-voltage inverter cell (HVC), is brought about by switching four lower-voltage inverter cells (LVC) each of 12 V. This relationship, which at first glance seems paradoxical, is based on the finding that, due to the quadratic relationship between the switching losses and the voltage value of the DC current supply of the inverter cell, switching the higher-voltage inverter cell (HVC) leads to a disproportionate loss that is able to be reduced by switching a larger number (namely in accordance with the voltage multiple) of lower-voltage inverter cells (LVC).

The modulator preferably interacts with a tap changer to which the reference signal is applied and that is designed to convert the reference signal into a voltage level signal that is applied to the modulator. This achieves reproducible and defined discretization of the reference signal that is used to drive the inverter cells of the multilevel inverter and specifies at least one voltage value (and generally also the curve form) of the generated AC voltage. It is thus possible per se to unambiguously determine, for each voltage level, how many higher-voltage inverter cells (HVC) and lower-voltage inverter cells (LVC) need to be switched. The number of higher-voltage inverter cells (HVC) to be switched thus corresponds for example to the integer component of a division of the voltage level by the rated voltage of the individual higher-voltage inverter cell (HVC), and the remainder defines the number of lower-voltage inverter cells (LVC) for fine adjustment.

It is expedient for the modulator to furthermore be designed to vary the number of higher-voltage inverter cells (HVC) to be switched based on at least one predefinable parameter and to determine a further number of the low-voltage inverter cells (LVC) to be switched and to switch these in an opposing manner for compensation purposes. According to this particularly advantageous aspect of the invention, for a given voltage to be output, the number of high-voltage inverter cells, on the one hand, and low-voltage inverter cells, on the other hand, to be switched on is not strictly predetermined, but rather should be varied selectively. The modulator thus provides a certain amount of ambiguity with regard to the number of higher-voltage inverter cells to be switched (for a given voltage), as a result of which the number of low-voltage inverter cells to be switched therefore also varies. For this variation when implementing a voltage signal to be output onto the number of higher-voltage or low-voltage inverter cells to be switched, the invention makes provision for the modulator, which varies the numbers, resulting from a strict determined implementation of the setpoint voltage signal, for the higher-voltage and low-voltage inverter cells. This variation may also be considered to be a distortion of the respective number. The modulator distorts the numbers, resulting from a strict deterministic division, of higher-voltage inverter cells (HVC) and lower-voltage inverter cells (LVC). It could therefore also referred to as a methodical distorter of the deterministic division.

An ambiguity resulting from such variation, which is alien to a normal implementation of a setpoint voltage signal in typical digital drive signals, entails a number of advantages:

Switching the higher-voltage inverter cells (HVC) makes it possible to quickly achieve a large voltage jump, whereas, using the low-voltage inverter cells (LVC), it is possible to achieve fine matching to the curve profile defined by the reference signal. Precise matching requires frequent switching of the inverter cells, but it is usually enough to switch the low-voltage inverter cells (LVC), actuation of which entails only few switching losses, to achieve this. The switching of higher-voltage inverter cells (HVC), which is considerably more expensive in terms of switching losses, is able to be reduced in terms of frequency by the modulator. One example of this is a switching rule in accordance with the pattern whereby, when an HVC cell is switched on once, it then remains on for as long as possible. This makes it possible to reduce switching losses in an efficient manner, as a result of which switching speed and therefore generally dynamic range is able to be increased. It will be understood that in this case the freedom for the variation by the modulator increases the more redundant inverter cells are present, in particular low-voltage inverter cells (LVC). The more of these are present, the longer switching of the higher-voltage inverter cells (HVC) is able to be delayed or avoided in the event of voltage changes. It is therefore expedient to provide redundant low-voltage inverter cells (LVC), that is to say more than defined per se by the ratio of the high to low voltage.

If for example the voltage of the higher-voltage inverter cells (HVC) is 48 V and the voltage of the low-voltage inverter cells (LVC) is 12 V, then instead of switching on one of the higher-voltage inverter cells (HVC), the same voltage increase may be achieved by switching on four of the low-voltage inverter cells (LVC). If the voltage increase is even 60 V, then it is also possible to avoid switching the higher-voltage inverter cell (HVC) when at least one redundant (fifth) low-voltage inverter cell (LVC) is available and is able to be switched on for this purpose. This accordingly applies vice versa in the case of negative voltages. This may furthermore also accordingly apply in cases in which a voltage decrease instead of a voltage increase is lined up. If for example a voltage of 72 V is output and if one higher-voltage inverter cell (HVC) and two low-voltage inverter cells (LVC) are switched on for this purpose, then, in order to reduce the voltage by 36 V, it is possible to avoid switching (that is to say switching off the already switched-on) higher-voltage inverter cell (HVC) by switching off two of the switched-on low-voltage inverter cells (LVC) and operating a third one with a negative voltage of 12 V, this then giving an overall voltage reduction of the desired 36 V, specifically without the higher-voltage inverter cell (HVC) (having higher switching losses) having to be switched for this purpose. This also applies when the 36 V voltage reduction takes place step by step. In the event of any change to the voltage level, the modulator decides whether the change should be made by switching an LVC or by switching an HVC and, in this example, three LVCs.

Provision is furthermore advantageously made that the modulator is actuated via an enable signal, and provision is made for a change detector that is designed to identify a change in the reference signal and to apply the enable signal to the modulator. The modulator, in the event of an unchanged reference signal, thus does not need to perform any changeovers, but rather these take place only when the reference signal changes anyway. This is identified by the change detector. This interacts with the modulator such that an enable signal is applied to the modulator in the event of an identified change. This reduces the activity of the modulator to cases in which a change in the reference signal occurs. As an alternative or in addition, provision may however be made that the change detector monitors the (voltage) level signal generated by a tap changer. This reduces the activity of the modulator to cases when the change in the reference signal is such that it also leads to a change in the (voltage) level signal.

The inverter cells expediently each have potential decoupling at output. The invention makes use of the fact here that AC voltage is by definition present at the output of the inverter cells, meaning that it is possible to achieve reliable potential isolation of the voltages ultimately output by the inverter cells using simple and inexpensive transformers in a manner requiring little outlay (in comparison with isolated individual DC voltage sources as would be required at input).

Provision is preferably furthermore made that the predefinable parameter comprises a switching frequency of the higher-voltage inverter cells (HVC), and the modulator is designed to minimize this switching frequency. It may furthermore be taken into consideration that, due to the higher voltage of the inverter cells (HVC), the power loss caused by switching is disproportionately greater. There is even a quadratic relationship, meaning that switching one of the higher-voltage inverter cells (HVC) brings about 16 times the switching power loss in comparison with switching one of the low-voltage inverter cells (LVC) at a voltage ratio of 4 to 1, as is the case for example for 48 V to 12 V. The overall power loss is able to be reduced considerably by reducing the switching frequency of the higher-voltage inverter cells (HVC). Provision is expediently made that the predefinable parameter comprises a metric for power loss of the inverter cells, and the modulator is designed to adapt power loss caused by actuating the higher-voltage inverter cells (HVC) to the power loss caused by actuating the low-voltage inverter cells (LVC). This is not only beneficial for efficiency, but also entails considerable advantages in relation to relieving thermal stress on the switching elements in the inverter cells.

Provision is furthermore expediently made that at least two alternative switching rules for voltage changes are implemented in the modulator, these both leading to the same voltage change but switching a different number of HVCs. In the event of voltage changes, the modulator may make a selection from these switching rules. Since these switching rules are predefined, an appropriate behavior of the modulator may be programmed using the switching rules. For a voltage increase of for example 12 V, one example of two such alternative switching rules “a)” and “b)” is that, according to switching rule a), the starting value of the low-voltage inverter cells (LVC) is increased by one level of 12 V and no change takes place in the higher-voltage inverter cells (HVC); or alternatively, according to switching rule b), the voltage output by the low-voltage inverter cells (LVC) is reduced by three levels with a total of 36 V and at the same time the output voltage of the higher-voltage inverter cells (HVC) is increased by one level, that is to say by 48 V—this ultimately means the desired increase by 12 V. Switching rule b) is obviously the more expensive one, since, when it is applied, as a result of switching one of the higher-voltage inverter cells (HVC), significantly higher switching losses arise than in the case of switching rule a). In order to achieve this, the switching rules are preferably implemented such that, in the event of a voltage increase, according to one of the alternative switching rules, the number of higher-voltage inverter cells (HVC) remains the same and one of the low-voltage inverter cells (LVC) is activated (switching rule a)), or, according to the other (switching rule b)) of the alternative switching rules, the number of switched higher-voltage inverter cells (HVC) is increased by one and a plurality of low-voltage inverter cells (LVC) are switched in an opposing manner, wherein this plurality corresponds to the voltage multiple minus one. This is the case in the event of a voltage increase by one level corresponding to a step down in the voltages able to be output by the low-voltage inverter cells (LVC). For larger voltage changes involving multiple such levels, the scheme implemented in switching rules applies accordingly.

This accordingly applies vice versa for the case of a voltage reduction. Provision is preferably made here that, according to one of the alternative switching rules, the number of higher-voltage inverter cells (HVC) remains the same and one of the low-voltage inverter cells (LVC) is deactivated, or, according to the other of the alternative switching rules, the number of switched higher-voltage inverter cells (HVC) is reduced by one and a plurality of low-voltage inverter cells (LVC) are switched in an opposing manner, wherein this plurality corresponds to the voltage multiple minus one. As already explained above for the voltage increase, this applies accordingly to larger voltage changes involving multiple levels.

Respective switching ranges are expediently assigned to the switching rules, wherein the switching ranges are preferably different for positive and negative polarity of the output voltage. This makes it possible to take into consideration the polarity of the voltage output by the inverter. Switching ranges define ranges of values for the switching rules. Provision is expediently made that, in the event of a positive output voltage, an increase in the higher-voltage inverter cells (HVC) should be delayed as long as possible. In the event of a negative value for the output voltage that increases slowly in the direction of zero, on the other hand, it is expedient to switch an increase in the higher-voltage inverter cells (HVC) as soon as possible, possibly taking hysteresis into consideration. In the event of a negative value for the output voltage that is further away from zero, on the other hand, it is expedient to postpone switching for as long as possible and instead to switch the low-voltage inverter cells (LVC). This thus switches off the higher-voltage inverter cells (HVC) again as early as possible, as soon as the voltage moves back in the direction of zero. This makes it possible to effectively counter the risk of saturation of the transformers of the higher-voltage inverter cells (HVC).

Provision is preferably optionally made for hysteresis in order to avoid unnecessarily frequent switching on and off of higher-voltage inverter cells (HVC) in the event of small voltage changes. It is particularly expedient, in the case of multiple changing between voltage increase and decrease, to block switching of the higher-voltage inverter cells (HVC), with preferably additional low-voltage inverter cells (LVC) being switched when the voltage increase or decrease exceeds the voltage value of the higher-voltage inverter cells (HVC).

Advantageously, limits of the switching ranges are dynamically changeable during operation, preferably on the basis of state variables of the higher-voltage inverter cells (HVC) and low-voltage inverter cells (LVC), in particular their respective switch-on time, magnetic flux and/or temperature. The operating state of the inverter cells and their components is thereby able to be taken into consideration. By way of example, the magnetic flux in the inverter cells may be monitored, in order in particular to avoid saturation of the inverter cells. This may take place such that a switching state for the outputting of positive voltage is selected such that it is precisely as long as the switching state for the outputting of negative voltage. This makes it possible to achieve a balance. This is particularly significant for the higher-voltage inverter cells (HVC), since they are by definition fed with a higher voltage and are (supposed to be) switched less often, this increasing the risk of uneven loading.

Provision may optionally be made for a monitoring unit that is designed to ascertain and to store magnetic flux in the higher-voltage inverter cells (HVC) and/or the low-voltage inverter cells (LVC). It is thus possible to monitor the magnetic flux in the respective inverter cells and to avoid saturation of the inverter cells by virtue of them then preferably being switched with an opposing voltage polarity until the magnetic flux has balanced out again. Provision may advantageously be made for a compensation unit that interacts with the monitoring unit and is designed such that, in the event of a voltage increase, out of the higher-voltage inverter cells (HVC) or low-voltage inverter cells (LVC), it first switches those with a low magnetic flux and, in the event of a voltage decrease, first switches those with a high magnetic flux. It is thus possible to perform compensation in an efficient manner, as a result of which it is possible to effectively prevent overloading of individual inverter cells and the accompanying risk of failure.

For this purpose, provision may furthermore advantageously be made for a switch-on time monitor for the higher-voltage inverter cells (HVC) or the low-voltage inverter cells (LVC), which ascertains the duration of a positive or negative voltage output of the HVCs and LVCs and, in the event of a respective presettable limit value being exceeded, deactivates the respective inverter cells (HVC or LVC). The dwell time of the individual inverter cells is thereby able to be controlled and limited effectively. If one of the inverter cells, and in particular the higher-voltage inverter cells (HVC) are relevant here, is switched on for too long, then it is not used but rather switched to idle mode, and another, less loaded one of the high-voltage inverter cells (HVC) is actuated instead.

Provision is preferably made for a control signal generator for the multilevel inverter, which is designed to generate a reference signal for driving the multilevel inverter, wherein the reference signal is a pattern for AC voltage to be output by the electrosurgical generator, in particular with regard to amplitude, frequency, curve form and/or duty cycle, wherein the curve form is preferably able to be set freely as desired. Using such a reference signal makes it possible to give a precise specification to the multilevel inverter in relation to the voltage to be generated and the voltage profile. This enables positive control of amplitude and curve form of the voltage that is output.

Provision is expediently made for a peak detector to which the reference signal is applied and that acts, with its output, on the modulator, in particular reduces or prevents switching of high-voltage inverter cells (HVC). This advantageously takes place such that, with the reference signal, information about the expected future value for the output signal of the multilevel inverter is available. This information signal concerning the future may be evaluated by the peak detector and be used to better drive the inverter cells. If it emerges, for example from a reference signal at a particular time, that the voltage increase is largely finished and the sinusoidal wave, after reaching its maximum, will soon recede, then the activation of a further higher-voltage inverter cell (HVC) may be blocked for the last steps of the voltage increase by the peak detector taking into consideration the voltage drop that will soon occur. Another one of the low-voltage inverter cells (LVC) (which causes low switching losses) may be actuated instead. This has the advantage that unnecessary switching procedures, in particular of the higher-voltage inverter cells (HVC), are able to be effectively avoided before the maximum is reached. In order to reach the peak voltage, an available (redundant) low-voltage inverter cell (LVC) is used instead.

The invention is explained in more detail below by way of example with reference to one advantageous embodiment. In the figures:

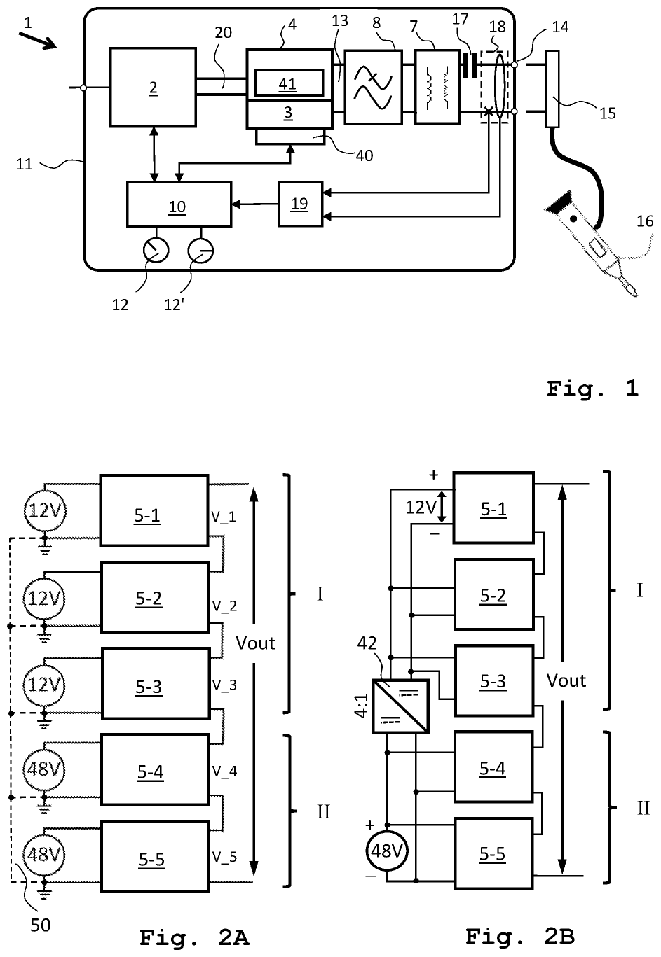

FIG. 1 shows a schematic illustration of an electrosurgical generator according to one exemplary embodiment with a connected electrosurgical instrument;

FIG. 2A, 2B show block diagrams of exemplary embodiments for a multilevel inverter of the electrosurgical generator according to FIG. 1 with cascaded inverter cells;

FIG. 3 shows a schematic circuit diagram of two of the inverter cells;

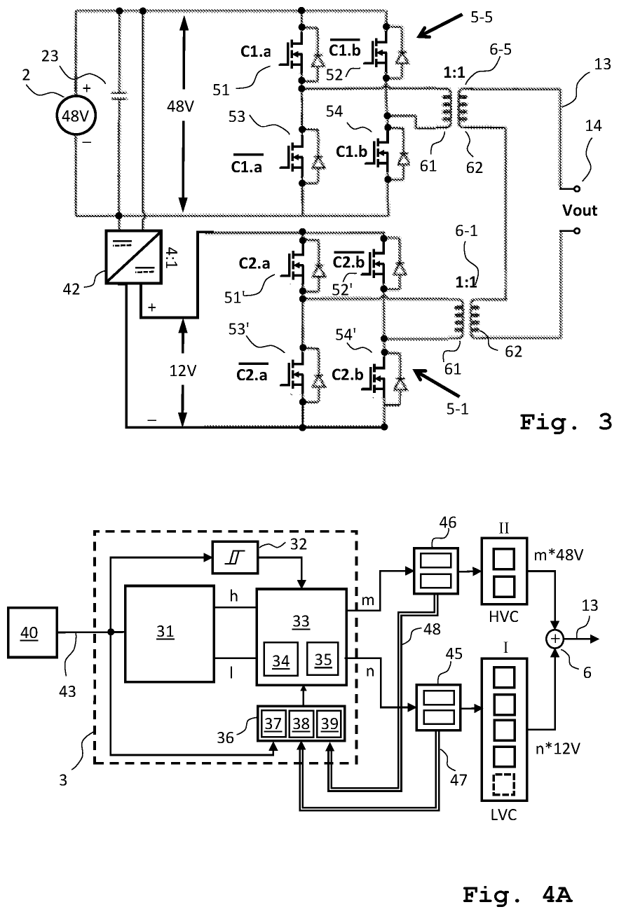

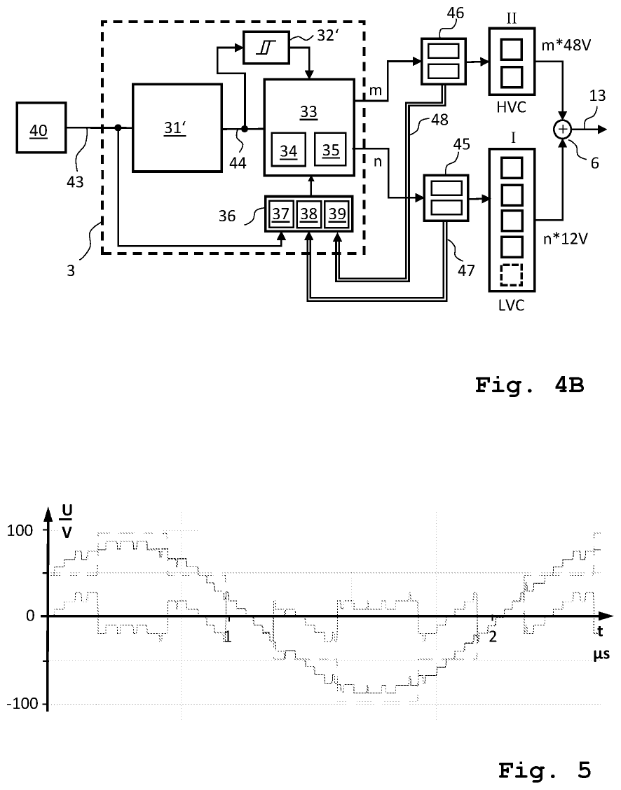

FIG. 4A, 4B show block diagrams of examples of a selector with a modulator for driving high-voltage and low-voltage inverter cells;

FIG. 5 shows a simplified example of the switching of higher-voltage and low-voltage inverter cells for implementing a reference signal by voltage level;

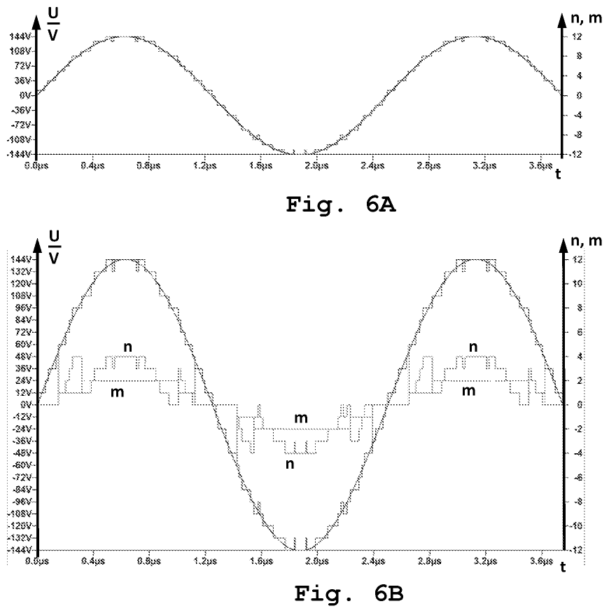

FIG. 6A, 6B shows another, more complex example of the switching of higher-voltage and low-voltage inverter cells for implementing a reference signal;

FIG. 7 shows a table containing switching rules for the modulator based on a polarity of the output voltage and a rise or fall in the reference signal;

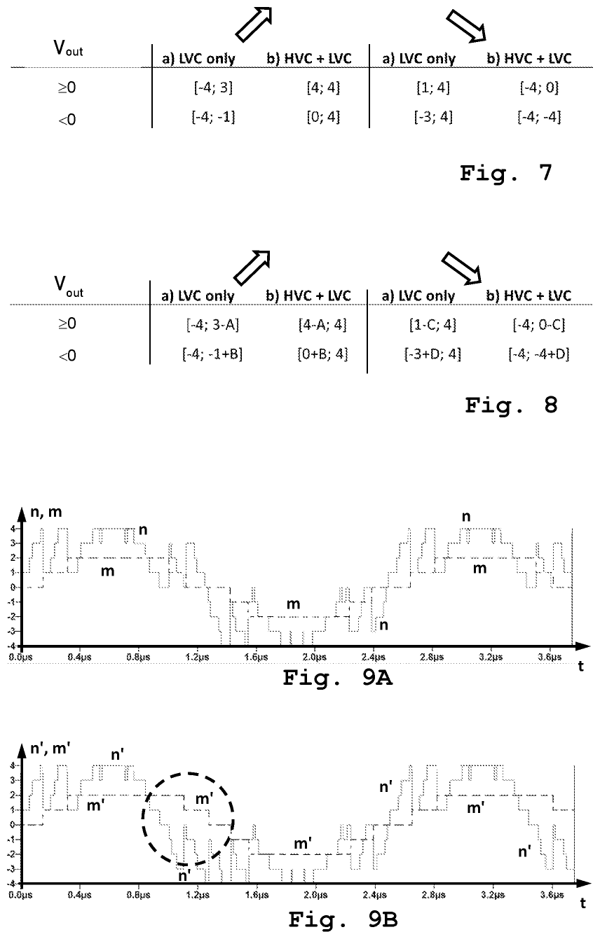

FIG. 8 shows a table containing changeable switching rules for the modulator as a variant to FIG. 7; and

FIG. 9A, 9B show exemplary switching profiles for high-voltage and low-voltage inverter cells, without and with considering magnetic saturation in the inverter cells.

An electrosurgical generator according to one exemplary embodiment of the invention is illustrated in FIG. 1. The electrosurgical generator, referenced in its entirety by the reference numeral 1, comprises a housing 11 that is provided with an output port 14 for an electrosurgical instrument 16; in the illustrated exemplary embodiment, this is an electrical scalpel. It is connected to the output port 14 of the electrosurgical generator 1 via a connection plug 15 of a high-voltage connection cable. The power output to the electrosurgical instrument 16 may be changed via a power controller 12.

In order to supply power to the electrosurgical generator 1, provision is made for a DC voltage supply 2, which is able to be connected, via a mains connection cable (not illustrated), to the public grid and is fed therefrom. The DC voltage supply 2 is a power supply unit in the illustrated exemplary embodiment. It comprises a rectifier and feeds a DC link circuit 20 with DC voltage, the value of which is preferably fixed and is for example 48 volts. However, it should not be ruled out that the DC voltage value is variable between 0 and around 400 volts, wherein the absolute value of the DC voltage may in particular depend on the set power, the type of electrosurgical instrument 16 and/or its load impedance, which in turn depends on the type of tissue being treated. However, an internal power supply unit is not necessary, meaning that the DC voltage supply may also be implemented by an external power supply unit, or provision is made for a direct DC feed, for example 24 volts in vehicles or 48 volts in stationary applications.

An inverter is fed by the DC link circuit 20 and generates, from supplied DC voltage, high-frequency AC voltage in the high-voltage range, at predefinable frequencies in the range between 200 kHz and 4 MHz. The inverter is designed in the structural form of a multilevel inverter 4, as will be explained in even more detail below. The frequency and curve form of the high-frequency AC voltage to be generated by the multilevel inverter 4 are in this case predefined by an inverter controller 41 on the basis of a reference signal 43 generated by a control signal generator 40 (see FIG. 4). The high-frequency AC voltage generated by the multilevel inverter 4 is routed via an output line 13, an output transformer 7 for stepping up the output voltage into the range of a few kilovolts and a low-pass filter 8 and, secured against undesirable DC current components by a blocking capacitor 17, output at the output port 14 for connection for the electrosurgical instrument 16. The voltage and current of the high voltage generated and output by the multilevel inverter 4 are furthermore measured by way of a combined voltage and current sensor 18, and the measured signals are supplied to a processing unit 19, which applies the corresponding data about the output voltage, current and power as feedback to an operating controller 10 of the electrosurgical generator 1, which for its part communicates with the control signal generator 40. The power controller 12 is also connected to the operating controller 10. The operating controller 10 is furthermore designed to set various what are known as modes, which are typically stored voltage/time profiles. Provision is made for a selection switch 12′ for the user to select the mode. The operating controller 10 furthermore interacts with the control signal generator 40, which is designed to generate the reference signal 43 for the AC voltage to be output, in particular with regard to amplitude, frequency, curve form and duty cycle and to output it to an inverter controller 41.

The multilevel inverter 4 comprises a plurality of series-connected inverter cells 5 that are driven by the inverter controller 41. The inverter cells 5 are divided into two groups I and II, which are fed, in groups, with DC voltage of different values. A first one is called “group I” and comprises low-voltage inverter cells (LVC), specifically three inverter cells 5-1, 5-2 to 5-3 in the example in FIG. 2. They are fed by a DC voltage source with a low DC voltage, 12 V in the example. A group of high-voltage inverter cells (HVC) is also formed, this being called “group II” and comprising two inverter cells 5-4, 5-5 in the example illustrated in FIG. 2, these being fed with a higher DC voltage, 48 V in the example. The voltages output by the two groups I and II are summed by way of a transformer 6 (see FIGS. 4A, 4B). Provision is furthermore made for a selector 3 that defines the inverter cells 5 to be driven, in particular the number “m” of high-voltage inverter cells (HVC) from group II to be driven and the number “n” of low-voltage inverter cells (LVC) from group I to be driven.

Reference is now made to FIG. 2A. In the exemplary embodiment illustrated there, a DC voltage source having a defined DC voltage is connected to the input (illustrated on the left in the drawing) of each of the inverter cells 5. The respective inverter cell 5 generates therefrom an AC voltage that is output at the output (illustrated on the right in the drawing) of the respective inverter cell 5 in the form of AC voltage. The number of inverter cells is not limited and is as desired per se. The inverter cells 5 are numbered consecutively in FIG. 2A with the designation “5-1”, “5-2” to “5-5”, wherein the number 5 is an example and any number of at least two inverter cells may be provided. The DC voltages applied at the input of the respective inverter cell 5 are optionally coupled in terms of potential via a busbar 50. The AC voltage output at the output of the respective inverter cell 5 is accordingly denoted “V_1”, “V_2” up to “V_5”. The series connection of the inverter cells 5 results in their output voltages being added, ultimately giving, as overall output voltage:

$${V}_{out} = {\sum }_{i = 1}^{N} V \_ i$$

Compared with a simple structure having only one DC voltage source, the outlay in terms of DC voltage sources is increased according to the invention, because more (in the example: two) than just one are now required. However, to make up for this, the number of voltage levels is increased considerably, specifically starting from eleven voltage levels with only one DC voltage source to more than twice that with 23 voltage levels. The number of voltage levels thus able to be achieved follows the formula

2*(mHVc*r+nLVc)+1,

wherein mHVc represents the number of higher-DC-voltage inverter cells (in the above example m=2), nLVc represents the number of low-DC-voltage inverter cells (in the above example n=3) and r represents the ratio of higher to low DC voltage (in the above example r=4).

The two DC voltage sources do not need to be isolated from one another in terms of potential, but rather they may share a common reference potential, as implemented in FIG. 2A by way of the busbar 50. This also makes it possible to generate the lower DC voltage from the higher DC voltage, which may be for example the DC voltage in the link circuit 20, by way of a DC-to-DC converter 42, in particular a DC-to-DC buck converter. In the present example, this would be designed for a ratiometric supply with a step-down ratio of 4:1, as illustrated in FIG. 2B. One advantage of this configuration by way of a ratiometric supply is that changes or fluctuations in the higher DC voltage are then reflected proportionally in the lower DC voltage, meaning that the relative gradation is maintained. This makes it possible for example to increase the output voltage by 12 V in two different ways: one conventionally by activating a further 12 V inverter cell, or by activating a 48 V inverter cell in combination with deactivating three 12 V inverter cells.

The structure of the individual inverter cells 5 and their interaction are illustrated by way of example in the schematic circuit diagram according to FIG. 3. A total of two inverter cells 5-1 and 5-5 are shown there in a cascaded arrangement, in order thus also to illustrate their supply with DC voltage of different values. The common DC voltage source 2, having a supply voltage Vin of 48 volts, is illustrated on the left-hand edge of the image. It is assigned a stabilization capacitor 23. The two inverter cells 5-3 and 5-4 are thereby supplied with DC voltage. Reference is first of all made below to the switching of the inverter cell 5-5, which is supplied with 48 volts. Provision is made for four power switches that operate as current valves and are arranged in an H-bridge configuration. The power switches are power semiconductor switches, for example configured as IGBTs, MOSFETs or GaNFETs. The power switches 51, 53 are connected in series and form a first branch, and the power semiconductors 52, 54 are likewise connected in series and form a second branch. The center taps of the two branches are guided out and connected to both ends of a primary winding 61 of a first transformer 6-5. The transformer 6-5 furthermore has a secondary winding 62, wherein the transformation ratio is 1:1 (it is pointed out that another transformation ratio may be provided, in particular in order to achieve pre-amplification, for example with a transformation ratio of 1:2). An output line 13 is connected to the secondary winding 62 and leads to the output port 14 of the electrosurgical generator 1 (possibly via a low-pass filter 8, not illustrated in FIG. 3, and an output transformer 7, see FIG. 1).

The two power switches 51, 53 of the first branch are driven by a common signal C1.a, wherein this signal is supplied to the power switch 53 in inverted form. The two power switches 52, 54 of the second branch are accordingly likewise driven by a common signal C1.b, wherein this signal is supplied to the power switch 52 in inverted form. The signals C1.a and C1.b are generated in a manner known per se by the inverter controller 41. This means that, in the event of a HIGH signal of C1.a, the power switch 51 is put into the on state and the power switch 53 is put into the off state, that is to say the first power branch applies a positive potential to the upper connection of the primary winding 61 of the transformer 6-1. Accordingly, in the event of a HIGH signal of C2.b, the power switch 54 is put into the on state, while the power switch 52 is put into the off state in the second power branch. The second power branch thus applies a negative potential to the lower connection of the primary winding 61. In the event of a LOW signal of C1.a or C1.b, this accordingly applies vice versa, that is to say the polarity at the primary winding 61 is reversed. An AC voltage is thus generated by the inverter cell 5-1 and applied to the primary winding 61 of the transformer 6-1.

The second inverter cell 5-1 has an identical structure, but is fed from the DC voltage source 2 via the ratiometric DC-to-DC converter 42, which brings about a reduction to a quarter of the input voltage. It thus outputs a DC voltage of 12 volts, which is supplied to the second inverter cell 5-1 in a manner identical per se to the first inverter cell 5-5. The same reference numerals are therefore used for identical elements in the figure. It is driven by way of control signals C2.a and C2.b that are generated by the inverter controller 41, for example by way of pulse width modulation (PWM), which is known per se, in a manner corresponding to what has been described above. It thus likewise outputs, at its output, an AC voltage that is applied to the primary winding 61 of a second transformer 6-1. Depending on the design of the DC-to-DC converter 42, the two inverter cells 5-1 and 5-5 are connected in terms of potential. This means that the AC voltages output directly by the inverter cells 5-1 and 5-5 are not readily able to be added, since they are linked to one another in terms of their potential. However, since this output AC voltage is supplied to each of the transformers 6-1 and 6-5, the AC voltages output by the transformers 6-1 and 6-5 are each potential-free and are readily able to be added to one another to give a common output voltage that is applied to the output line 13. If the inverter cells 5-1 and 5-5 are however decoupled in terms of potential through an appropriate design of the DC-to-DC converter, then the output AC voltages may also be added directly through a series connection without this transformer.

The overall voltage generated and summed in this way (and by other inverter cells 5-2 to 5-4) is output via the output line 13, at the end of which the low-pass filter 8 is arranged. This may be designed for example as a second-order filter comprising an inductor and a capacitor. It is pointed out that stray inductances of the transformers 6-1 to 6-n also contribute to the inductance of the low-pass filter, and may possibly at least partially replace the inductor. The low-pass filter 8 is tuned such that interference in the generated AC voltage due to the switching frequency of the power switches in the inverter cells 5 of the multilevel inverter 4 is filtered out. The output of the low-pass filter 8 is applied to a primary winding of the output transformer 7, which brings about galvanic isolation of the output port 14 connected to the secondary winding. Provision is furthermore made for a blocking capacitor 17. This serves as a safety element for preventing the output of DC current components to the surgical instrument 16.

The control signal generator 40 generates, in particular on the basis of specifications for the operating voltage 10, a reference signal 43 for driving the multilevel inverter 4. This is an AC voltage signal that is typically sinusoidal and has a particular frequency and amplitude. Reference is now made to FIGS. 4A, 4B. The reference signal 43 is applied to the selector 3, which determines therefrom the number and the type (HVC or LVC) of the inverter cells 5 to be driven, specifically broken down by low-voltage inverter cells (LVC) from group I and higher-voltage inverter cells (HVC) from group II. For this purpose, the selector 3 comprises a tap changer 31 and a modulator 33. The tap changer 31 is designed to convert the typically continuous reference signal 43 into a voltage level signal. This is a discrete signal that is indicative of the number of voltage levels, specifically typically expressed in levels the value of which results from the voltage of the low-voltage inverter cells (LVC) from group I, that is to say in the present example in levels of 12 V. One example of such a continuous reference signal 43 and a level signal formed therefrom expressed in levels of 12 V is depicted in FIG. 6A. The graduated curve shows the voltage level signal and thus forms a discretization of the reference signal 43 represented by the continuous curve.

The tap changer 31 may furthermore already make a preliminary division as to what portion thereof is incumbent on the low-voltage inverter cells (LVC) from group I or on the higher-voltage inverter cells (HVC) from group II. In one embodiment, as illustrated in FIG. 4A, this may be achieved for example by minimizing the basic number of inverter cells required to achieve the voltage in accordance with the reference signal 43. Such a division may comprise two signals, a signal “h” for the basic number of higher-voltage inverter cells (HVC) from group II to be switched on and a signal “1” for the basic number of low-voltage inverter cells (LVC) from group I to be switched on. This may in particular be a purely numerical division, for example whereby, in order to generate a voltage of 84 V, exactly one higher-voltage inverter cell (HVC) from group II and three low-voltage inverter cells (LVC) from group I need to be driven.

However, these basic numbers “h” and “1” are not used directly for drive purposes, but rather are varied by way of the modulator 33. The modulator 33 is intended to reduce the switching frequencies of the high-voltage inverter cells (HVC) in group II. As a replacement for this, low-voltage inverter cells (LVC) in group I are switched instead. This is described in more detail below. The resultant division by the modulator 33 into the numbers “n” for the low-voltage inverter cells (LVC) from group I to be switched and “m” for the higher-voltage inverter cells (HVC) from group II to be switched differs depending on the situation and is ambiguous according to the invention.

The number “n”, which is thus varied, of low-voltage inverter cells (LVC) from group I to be switched and the varied number “m” are output as output signals from the modulator 33 and applied to subcontrollers 45, 46 for the inverter cells LVC from group I or HVC from group II. These control the respective inverter cells LVC in group I or HVC in group II in a manner known per se. The subcontrollers 45, 46 acquire switching data regarding the individual inverter cells 5 of the low-voltage inverter cells (LVC) from group I or the high-voltage inverter cells (HVC) from group II. These data comprise, inter alia, switch-on time, counts of the number of switching procedures and the magnetic flux through the individual inverter cells 5 and their transformer 6. They transmit corresponding state data via data lines 47, 48 to the modulator 33 and/or an adaptation module 36 upstream thereof.

The modulator 33 does not necessarily need to operate continuously. It may be enough for it to be actuated and to divide the voltage level signal into the number of low-voltage inverter cells (LVC) and high-voltage inverter cells (HVC) to be switched in particular when a change has resulted in the voltage level signal or in the reference signal 43. To this end, provision is optionally made for a change detector 32, which monitors the reference signal 43 and actuates the modulator 33 in the event of a change.

In one alternative embodiment, as illustrated in FIG. 4B, the tap changer 31′ has a different design, such that it outputs only a discretized reference signal (level signal) 44. The modulator 33 determines directly therefrom the number “m” of high-voltage inverter cells (HVC) to be switched and the number of low-voltage inverter cells (LVC) to be switched. This is explained with reference to a simplified example: The tap changer 31′ generates a discrete level signal 44 from the reference signal 43. The modulator 33 is designed to compare the level signal 44 with the last previous value of the level signal. The comparison may reveal that there is a rise, a fall or constancy. This is detected by way of the change detector 32′. The values for the numbers “m” and “n” are adjusted only in the event of a rise or a fall, that is to say only when a change has occurred in comparison with the previous level signal. This is achieved by way of the switching rules, as are explained further below with reference to the examples illustrated in FIGS. 7 and 8.

One example of the voltage generation using higher-voltage inverter cells (HVC) is illustrated in FIG. 5 by a dashed line, and the voltage generated by the low-voltage inverter cells (LVC) is illustrated by the solid line close to the zero line. Together, they give the desired sinusoidal profile, as illustrated by the quantized sinusoidal line. It may be seen that each of the two higher-voltage inverter cells (HVC) from group II needs to be switched on and off just once per half-wave, and the further adjustment is performed by frequently switching the low-voltage inverter cells (LVC) from group I. In this case, the LVCs from group I both increase the voltage (for example right at the start in the interval from 0 to 0.25 μs) and also reduce it through compensatory counter-switching in order to reduce the temporally excessively high voltage output by the inverter cells HVC (for example in the interval 0.25 to 0.65 μs and 0.87 to 0.98 μs). Switching procedures of the higher-voltage inverter cells (HVC) are thereby able to be avoided and the number thereof is thus able to be reduced, and the considerable switching power loss arising as a result of the switching of the HVC cells is thus also able to be reduced.

A more complex example of more inverter cells is illustrated in FIG. 6B. The values “m” resulting here for the number of higher-voltage inverter cells (HVC) from group II to be switched on are illustrated by a dashed line and for the number of low-voltage inverter cells (LVC) from group I to be switched on are illustrated by a solid graduated line “n”. It may clearly be seen on the profile of the line denoted “m” that the switching activity of the higher-voltage inverter cells (HVC) is considerably reduced, in particular in the region of the amplitude maxima of the reference signal and the zero crossing. By prioritizing switching activities of the low-voltage inverter cells (LVC), the higher-voltage inverter cells (HVC), having higher switching losses, are able to be spared.

To achieve this, switching rules 34 are implemented in the modulator 33. The switching rules are based on an exemplary configuration having two higher-voltage inverter cells (HVC) in group II and 4 low-voltage inverter cells in group I, as also illustrated in FIGS. 4A, 4B. Using the change detector 32, the switching states of the inverter cells are changed only when the reference signal 43 has also changed. The switching rules 34 provide two possible alternatives for the case of a rise:
a) increasing the voltage output by the low-voltage inverter cells (LVC) by 1 level (corresponding to 12 V) without any change regarding the high-voltage inverter cells (HVC); or
b) reducing the voltage output by the low-voltage inverter cells (LVC) by 3 levels (corresponding to −36 V) and increasing the voltage output by the high-voltage inverter cells (HVC) by one level (+48 V).

Both alternatives a), b) lead to the same voltage change by one level, specifically by +12 V. Alternative b) requires switching of one of the high-voltage inverter cells (HVC), which, due to the quadratic relationship with the voltage supply multiplied by four, means 16 times more switching losses in comparison with one of the low-voltage inverter cells (LVC). This is added to by another three switching procedures of the low-voltage inverter cells (LVC). Alternative b) thus means 19 times more energy loss than alternative a) of the switching rules 34.

These relationships are taken into consideration by the switching rules 34 illustrated in FIG. 7. Reference is now made to the left-hand column, which concerns the case of a voltage rise. This contains the two alternatives a) and b). Input parameters are the polarity of the output voltage and the voltage output by the low-voltage inverter cells (LVC) from group I, expressed in voltage levels of the LVCs. In this case, “1” represents an output voltage of +12 V, “4” represents an output voltage of +48 V, and accordingly “−4” represents an output voltage −48 V. The switching rules 34 implemented in the modulator 33 then state, for a positive polarity of the output voltage, that, in the event of a voltage level between −4 and 3 (corresponding to −48 V to +36 V) for group I, switching rule a) is used, that is to say the voltage output by the LVC converter cells from group I is increased by 12 V. If however voltage level 4 is already present in group I, then alternative switching rule b) is used, in which the HVC inverters from group II are switched up by one level, resulting in an increase by 48 V, and the LVC inverter cells from group I are switched down by three levels for compensation purposes, corresponding to −36 V, ultimately resulting in the desired increase by 12 V. If the polarity of the output voltage is negative, then appropriately adjusted switching ranges of −4 to −1 for alternative a) and 0 to 4 for alternative b) of the switching rules apply. The appropriate switching rule for the case of a voltage drop is illustrated in the right-hand column of FIG. 7. Alternatives a) and b) apply in this case too, but with adjusted ranges as may be seen from FIG. 7.

The modulator 33 may furthermore comprise a hysteresis module 35. This is designed to acquire the switching frequency in relation to the high-voltage inverter cells (HVC) from group II and to minimize their switching procedures in the event of excessive switching activity. For this purpose, the hysteresis module 35 acts for example on the switching rules 34 so as to change the range limits such that alternative b) becomes rarer.

The switching rules 34 and their switching ranges may be adapted by an adaptation module 36, in particular on the basis of operating conditions of the multilevel inverter 4 with its inverter cells 5. The adaptation module 36 comprises a monitoring unit having a compensation unit 38. It detects the magnetic flux in the individual inverter cells in each of groups I and II and thus acts on the switching ranges of the switching rules 34. The switching ranges of the switching rules 34 may thereby be changed dynamically. If the overall magnetic flux through the high-voltage inverter cells (HVC) is too high, then the switching ranges are changed such that these cells are switched on only later and that they are switched off again earlier. Parameters B and D may be changed for this purpose, as illustrated in the modified switching rules 34 according to FIG. 8. If by contrast, on the other hand, the magnetic flux is too low, then parameters A and C may be used to change the switching ranges such that the high-voltage inverter cells (HVC) are switched on earlier and switched off again only later. It is thereby possible to balance out the magnetic flux and avoid saturation.

The adaptation module 36 furthermore comprises an optional switch-on time monitor 39. This acquires, separately for the low-voltage inverter cells (LVC) and the high-voltage inverter cells (HVC), the duration of a positive or negative voltage output. If particular preset limit values are exceeded, then the switching ranges may be adjusted dynamically in a manner similar to that described above for magnetic saturation. Provision may however also be made that the corresponding highly loaded inverter cells are switched off for a certain time.

The effect of the adaptation module 36 with the dynamically changed switching ranges is illustrated in FIGS. 9A and 9B. FIG. 9A shows, as a starting point, the switching behavior in accordance with the switching rules 34 with unchanged switching ranges, as illustrated in FIG. 7. If the compensation unit 38 identifies that the magnetic flux through the high-voltage inverter cells (HVC) in group II is too low, then the adaptation module 36 shifts the parameter C, for example by a value of 3. This then results in appropriately changed switching ranges in accordance with the modified switching rules, as illustrated in FIG. 9B with the parameter C=3. This means that the high-voltage inverter cells (HVC), when they have been switched on once, remain switched on for longer, that is to say switch-off is delayed, as shown by the line m′. There is no resultant effect on switch-on here (this could be achieved by changing the parameter A). The switching activity of the low-voltage inverter cells (LVC) changes accordingly, as shown by the line n′. As a result, the switch-on and switch-off behavior of the high-voltage inverter cells (HVC) is thus asymmetric in the sense that they switch off considerably later. They are thus switched on for longer, which increases their magnetic flux. Dynamically changing the switching range of the switching rules 34 thus achieves the desired aim of increasing the magnetic flux in the high-voltage inverter cells (HVC).

Provision is furthermore made for an optional peak detector 37. The reference signal 43 is applied thereto. It is designed to identify the occurrence of signal peaks in the reference signal 43, for example when the amplitude reaches its maximum value. If this is identified, then the peak detector 37 may act on the modulator 33 such that switching, due per se in accordance with the switching rules 34, of high-voltage inverter cells (HVC) is blocked, and instead a surplus low-voltage inverter cell (LVC) is switched in order to achieve the last voltage levels. Such an optional surplus LVC inverter cell in group I is illustrated by a dashed line in FIG. 4. It is thereby possible to make use of the fact that the amplitude maximum is known and simple thanks to the reference signal 43. The peak detector 37 identifies this and acts on the modulator 33 so as to block or at least to minimize the switching procedures of high-voltage inverter cells (HVC) 5 close to the amplitude maxima.

1. An electrosurgical generator that is designed to output a high-frequency AC voltage to an electrosurgical instrument, comprising a DC voltage supply and a high-voltage inverter that is fed from the DC voltage supply and generates a high-frequency AC voltage having a variable voltage and frequency that is applied to an output for connection of the electrosurgical instrument, wherein
the inverter is designed as a multilevel inverter controlled by a reference signal for the voltage to be output and having at least two groups of series-connected inverter cells, wherein each group is supplied with a different DC voltage and wherein voltages output by the groups are summed to be output at the output.

2. The electrosurgical generator as claimed in claim 1, wherein the inverter cells are combined into two groups, of which a first group are supplied with a lower DC voltage than a different, second group.

3. The electrosurgical generator as claimed in claim 1, wherein the inverter cells have a bipolar configuration and output at least three different output voltage levels, which are positive, negative or zero.

4. The electrosurgical generator as claimed in claim 1, wherein there is a fixed ratio between the absolute value of the voltage that is generated by the inverter cells of a second group and the absolute value of the voltage that is generated by the individual inverter cells of a first group.

5. The electrosurgical generator as claimed claim 1, wherein, in order to drive the inverter cells of the groups, provision is made for a modulator that is designed to reduce switching frequencies of high-voltage inverter cells by selectively replacing actuation of the high-voltage inverter cells with actuation of a plurality of de-low-voltage inverter cells.

6. The electrosurgical generator as claimed claim 1, wherein a tap changer interacts with a modulator, to which tap changer the reference signal is applied and which tap changer is designed to convert the reference signal into a voltage level signal that is applied to the modulator.

7. The electrosurgical generator as claimed in claim 5, wherein the modulator is actuated via an enable signal, and provision is made for a change detector that is designed to identify a change in the reference signal and/or voltage level signal and to apply the enable signal to the modulator.

8. The electrosurgical generator as claimed in claim 5, wherein the modulator is furthermore designed to vary the number of inverter cells of the second group to be switched based on at least one predefinable parameter and to determine a further number of the inverter cells of the first group to be switched and to switch these in an opposing manner for compensation purposes.

9. The electrosurgical generator as claimed in claim 8, wherein the predefinable parameter comprises a switching frequency of the inverter cells of the second group, and a modulator is designed to minimize this switching frequency.

10. The electrosurgical generator as claimed in claim 8, wherein the predefinable parameter comprises a metric for power loss of the inverter cells, and the modulator is designed to adapt power loss caused by actuating the inverter cells of the second group to the power loss caused by actuating the inverter cells of the first group.

11. The electrosurgical generator as claimed in claim 5, wherein alternative switching rules for voltage changes are implemented in the modulator, these both leading to the same voltage change but switching a different number of inverter cells of the second group.

12. The electrosurgical generator as claimed claim 11, wherein, in the event of a voltage increase,
according to one of the alternative switching rules, the number of inverter cells of the second group remains the same and one of the inverter cells of the first group is activated, or
according to the other of the alternative switching rules, the number of switched inverter cells of the second group is increased by one and a plurality of inverter cells of the first group are switched in an opposing manner, wherein this plurality corresponds to the voltage multiple minus one.

13. The electrosurgical generator as claimed in claim 11, wherein, in the event of a voltage decrease,
according to one of the alternative switching rules, the number of inverter cells of the second group remains the same and one of the inverter cells of the first group is deactivated, or
according to the other of the alternative switching rules, the number of switched inverter cells of the second group is reduced by one and a plurality of inverter cells of the first group are switched in an opposing manner, wherein this plurality corresponds to the voltage multiple minus one.

14. The electrosurgical generator as claimed in claim 11, wherein respective switching ranges are assigned to the switching rules, wherein the switching ranges are different for positive and negative output voltage polarity.

15. The electrosurgical generator as claimed in claim 14, wherein limits of the switching ranges are dynamically changeable during operation, magnetic flux and/or temperature.

16. The electrosurgical generator as claimed in claim 5, wherein the modulator is designed to block switching of the inverter cells of the second group, with additional inverter cells of the first group being switched when the voltage increase or decrease exceeds the voltage value of the inverter cells of the second group.

17. The electrosurgical generator as claimed in claim 1, wherein provision is made for a monitoring unit that is designed to ascertain and to store magnetic flux in the inverter cells of the second group and/or the inverter cells of the first group.

18. The electrosurgical generator as claimed in claim 17, wherein provision is made for a compensation unit that interacts with the monitoring unit and is designed such that, in the event of a voltage increase, it first switches inverter cells of the second group or inverter cells of the first group with a low magnetic flux and, in the event of a voltage decrease, first switches those of the inverter cells with a high magnetic flux.

19. The electrosurgical generator as claimed in claim 1, wherein
provision is made for a control signal generator for the multilevel inverter, which is designed to generate a reference signal for driving the multilevel inverter, wherein the reference signal is a pattern for AC voltage to be output by the electrosurgical generator, wherein the curve form is able to be set freely as desired.
